(19) United States
(12) Patent Application Publication
(10) Pub. No.: US 2008/0161268 A1
(43) Pub. Date: Jul. 3, 2008

(54) QUATERNARY AMMONIUM SALT ANTIBACTERIAL AND WATER-BORNE COATING MATERIAL HAVING ANTIBIOTIC PROPERTY CONTAINING THE SAME

(75) Inventors: Cheng-Liang Yen, Taipei City (TW); Pei Tien, Taipei City (TW); Ya-Chi Huang, Taipei City (TW); Chi Hua Huang, Taipei City (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, omitted

(73) Assignee: COMPAL ELECTRONICS, INC., Taipei City (TW)

(21) Appl. No.: 11/829,085

(22) Filed: Jul. 27, 2007

(30) Foreign Application Priority Data
Dec. 28, 2006 (TW) 95149446

(51) Int. Cl. C09D 5/00; A61K 31/14; A61K 31/695; A61Q 19/00; C07C 215/40; C07F 7/10

(52) U.S. Cl. 514/63; 514/642; 523/122; 556/413; 564/291

(57) ABSTRACT

A quaternary ammonium salt antibacterial is provided, which has a structural formula as follows:

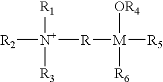

in which M represents, for example, Si; R represents a single bond or a C₁-C₄ alkyl group; R₁-R₃ are the same or different to each other and represent a C₃-C₁₈ alkyl group, respectively; R₄ represents a C₁-C₈ alkyl group or hydrogen; R₅-R₆, are the same or different to each other and represent a C₁-C₈ alkyl group, alkoxy group, or hydroxyl group, respectively; and X represents halogen.

### CROSS-REFERENCE TO RELATED APPLICATION

This application claims the priority benefit of Taiwan application serial no. 95149446, filed Dec. 18, 2006. All disclosure of the Taiwan application is incorporated herein by reference.

### BACKGROUND OF THE INVENTION

1. Field of the Invention

The present invention relates to an antibacterial and the application thereof. More particularly, the present invention relates to a quaternary ammonium salt antibacterial and the application thereof.

2. Description of Related Art

Antibacterials are new functional aids having bacteriostatic and bactericidal properties. Generally, an antibacterial material, for example, antibacterial coating material, antibacterial plastic, and antibacterial fiber, is obtained by adding an antibacterial having antibacterial ingredients into an applied material. As antibacterial materials have been used in a wide application range in a large amount in daily life, the development speed of the development and application of antibacterial materials is very fast. Therefore, the antibacterial materials have significant meaning in improving the living environment of human being and reducing the risk of diseases.

Action mechanism of antibacterials includes the following aspects: (1) interfering the synthesis of cell wall, (2) damaging cell membrane, (3) inhibiting the synthesis of protein, and (4) interfering the synthesis of nucleic acids.

(1) Interfering the synthesis of cell walls: a primary component of the cell wall of bacteria is peptido glycan. The interference effect of the antibacterial on the cell wall is mainly realized by inhibiting the cross linking of glycan chain and peptide, thus the integrity of the cell wall is lost, and the protection against osmotic pressure is reduced, resulting in the damage and death of the bacteria body.

(2) Damaging cell membranes: the cell membranes is damaged and destroyed, resulting in the death of bacteria to die.

(3) Inhibiting the synthesis of protein: the synthesis process of protein is altered and terminated to kill the bacteria.

(4) Interfering the synthesis of nucleic acids: the replication of genetic information is blocked, which includes, for example, the synthesis of DNA, RNA, and transcription of mRNA by DNA template.

The antibacterials in the antibacterial material can mainly be divided into three types: natural type, inorganic type and organic type. The natural antibacterials are prepared by antibiotic ingredients contained in natural animals and plants, such as chitosan and concentrated sorbic acid extracted from crustaceans, which have the advantages of low toxicity. However, the natural antibacterials have the disadvantages of short lifetime, poor heat resistance, and narrow application range. The natural antibacterials are mainly applied to disposal plastic product, such as food packaging pouch.

Inorganic antibacterials generally contain metal ion ingredient such as silver, zinc, and copper and inorganic carrier such as zeolite, phosphate, and hydroxyapatite. The antibiotic persistency is enhanced by the release action. The inorganic antibacterials have the advantages of having no drug resistance generated, and excellent heat resistance, and at the same time, have the disadvantage of high cost.

Organic antibacterials take organic acids, phenols, alcohols, and quaternary ammonium salts as main ingredients. The antibiotic mechanism of the organic antibacterials includes damaging the cell membrane, to denature the proteins or block the metabolism. The organic antibacterials have the advantages strong bactericidal power and wide source, and the disadvantages are high toxicity, generation of microbial drug resistance, and poor heat resistance. Currently, the organic antibacterials are mainly applied in coating material and plastics (such as flexible polyvinyl chloride, and polyethylene) having low processing temperatures.

Among the organic antibacterials, the sterilizing effect mechanism of the antibiotic mechanism of quaternary ammonium salt has two possible ways. One of the ways include combining the quaternary ammonium salt and the bacteria by utilizing the property that the positive ions of the quaternary ammonium salt itself is likely to combine with negative ions of the bacteria; then, damaging the cell membrane or the cell wall of the bacteria by the long alkyl chain contained in the quaternary ammonium salt, to kill the bacteria. The other way includes the processes as described previously, but the antibacterial effect is realized by contacting the bacteria with the antibacterial, so the constitutive property of the bacteria side is weakened, thus rupturing of the cell wall or the cell membrane. Therefore, the content in the bacteria body is leaked, so that the bacteria can not survive and been killed.

Due to the property that the positive ions of the quaternary ammonium salt itself is likely to combine with the negative ions of the bacteria, the quaternary ammonium salt can have high bactericidal power, and at the same time, the positive ions of the quaternary ammonium salt and the negative ions in the paints or surfactants attract each other. Therefore, the quaternary ammonium salt will be precipitated in the paints or surfactants having negative ions, thus the application of the quaternary ammonium salt is usually limited.

### SUMMARY OF THE INVENTION

Accordingly, the present invention is directed to a quaternary ammonium salt antibacterial, which can be dissolved in paints or surfactants having negative ions to exert antibacterial effect efficiently.

The present invention provides a quaternary ammonium salt antibacterial, which has a structural formula as follows:

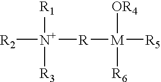

in which M includes Si; R represents a single bond or a C₁-C₄ alkyl group; R₁-R₃ are the same or different to each other and represent a C₃-C₁₈ alkyl group, respectively; R₄ represents a C₁-C₈ alkyl group or hydrogen; R₅-R₆ are the same or different to each other and represent a C₁-C₈ alkyl group, alkoxy group, or hydroxy group, respectively; and X represents a halogen.

The present invention further provides a lotion containing the quaternary ammonium salt antibacterial.

The present invention still provides a water-borne coating material having antibiotic property, which contains 10-90 wt % of resin, 10-90 wt % of water, and 10-20000 ppm of quaternary ammonium salt antibacterial, which has a structural formula as follows:

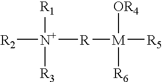

in which M represents, for example, Si; R represents a single bond or a C₁-C₄ alkyl group; R₁-R₃ are the same or different to each other and represent a C₃-C₁₈ alkyl group, respectively; R₄ represents a C₁-C₈ alkyl group or hydroxyl group; R₅-R₆ are the same or different to each other and represent a C₁-C₈ alkyl group, alkoxy group, or hydrogen, respectively; and X represents a halogen.

According to examples according to the present invention, the water-borne coating material having antibiotic property further contains an additive with a content of about 0.1-50 wt %.

According to the examples according to the present invention, the pH value of the water-borne coating material having antibiotic property is about 2-9.

The quaternary ammonium salt antibacterial of the present invention can be dissolved in paints, lotions, or surfactants having negative ions to exert the antibacterial effect efficiently.

In order to make the aforementioned and other objects, features and advantages of the present invention comprehensible, preferred embodiments accompanied with figures is described in detail below.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 shows the results of potential measurement according to an embodiment of the present invention.

### DESCRIPTION OF EMBODIMENTS

The present invention provides a quaternary ammonium salt antibacterial, which has a structural formula as follows:

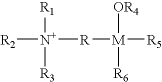

in which M represents, for example, Si; R represents a single bond or a C₁-C₄ alkyl group; R₁-R₃ are the same or different to each other and represent a C₃-C₁₈ alkyl group, respectively; R₄ represents a C₁-C₈ alkyl group or hydrogen; R₅-R₆ are the same or different to each other and represent a C₁-C₈ alkyl group, alkoxy group, or hydroxy group, respectively; X represents a halogen.

R represents a single bond or a C₁-C₄ alkyl group, such as methylene (CH₂), ethylidene (C₂H₄), propylidene (C₃H₆), and butylidene (C₄H₈).

The examples of R₁-R₃ representing a C₃-C₁₈ alkyl group include propyl (C₃H₇), butyl (C₄H₉), pentyl (C₅H₁₁), and hexyl (C₆H₁₃).

The examples of R₅-R₆ representing a C₁-C₈ alkyl group include methyl (CH₃), ethyl (C₂H₅), propyl (C₃H₇), and butyl (C₄H₉). The examples of R₅-R₆ representing a C₁-C₈ alkoxy group include methoxy, ethoxy, propyloxy, and butoxy.

X represents a halogen, such as fluorine, chlorine, bromine, and iodine.

N+ in the quaternary ammonium salt of the present invention can make the bacteria charges non-uniform, resulting in the rupture and death of bacteria. At least one of R₁-R₃ in the quaternary ammonium salt is a C₃-C₁₈ long alkyl chain, which can pierce the cell wall of the bacteria as a spear. Thereby, the cell wall is ruptured, and the intercellular substances stream out, which leads to the death of bacteria. OR₄ can be an alkoxy group which can be hydrolyzed into an OH or a hydroxylgroup. According, the quaternary ammonium salt antibacterial can be dissociated by appropriately controlling the pH value. The structure after the dissociation has the same charge repulsion force as the negative ion, so that the antibacterial is stable in paints or surfactants having negative ions to produce an effective antibacterial effect.

When M in the quaternary ammonium salt antibacterial having above-described the structural formula is Si, R₄ and R₅ are an alkoxy OR′, the siloxane structure (Si—OR′) will produce Si—OH through hydrolysis. The isoelectric point of Si—OH is at pH=2, thus, the organic quaternary ammonium salt having the siloxane structure can be dissociate into Si—O⁻ by adjusting the pH value to be greater than or equal to 2 and less than or equal to ≦9, and the reaction mechanism is as follows:

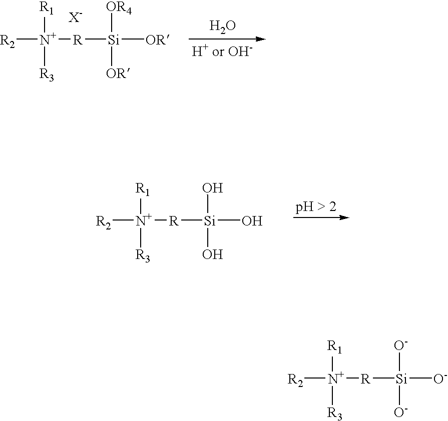

The structure after the dissociation has the same charge repulsion force as the negative ion, so that the antibacterial is stable in paints or surfactants having negative ions to produce an effective antibacterial effect.

The antibacterial of the present invention can be dissolved in the water phase or the solvent phase of a coating material, such as, an oil-borne coating material or a water-borne coating material. The pH value of the coating material is related to the isopotential of the antibacterial, such as 2-9.

When the quaternary ammonium salt antibacterial is applied in a latex paint, ingredients of the latex paint further include a resin, an additive, a pigment and a solvent, as well as a diluent.

The content of the quaternary ammonium salt antibacterial in the latex paint is 10-20000 ppm.

The resin in the latex paint is a major component of the coating material for forming a coating film, which is also a curing agent for fixing the pigment to surfaces of the coated object. The content of the resin is 10-90 wt %. The categories of the resin include emulsified acrylic resin, emulsified polyurethane (PU), ethylene-vinyl acetate copolymer (EVA) resin, vinyl chloride-vinyl isobutyl ether copolymer resin.

The additive in the latex paint is a minor component of the coating film for improving the quality of the coating material. The additive has the functions of preventing the generation of coating material defects during production, storage, and coating processes. The categories of the additive include a dispersant, a drying agent, a glazing agent, a planarizing agent, an antifoaming agent, and an anti-precipitation agent. The content of the additive is 0.1-50 wt %.

The pigments in the latex paint have the functions of providing colors of the coating material and protection. The pigments are generally divided into three groups: body pigments, anti-rust pigments, and coloring pigments. The body pigments include mica powder, calcium carbonate, and talc, and so on, and have the effects of enhancing adhesive property, weather resistance, and intensity of the paint. The anti-rust pigments include red lead powder, zinc powder, and zinc chromate, and so on, and have the effect of preventing the surfaces of the metal from being eroded. The coloring pigments are divided into organic pigments and inorganic pigments, in which the inorganic pigment are predominating, include titanium white powder, carbon black, iron oxide, and cadmium pigment, and so on, and have the effects of enhancing the hiding power, and the coloring power of the coating material. The content of the pigment is 1-30 wt %.

The solvent and the diluent in the latex paint are added for the resins have high viscosity, which is disadvantageous for grinding and the subsequent adjustment of the mixing of ingredients, and is difficult to be coated during operation. Accordingly, the solvent and the diluent do not only has the functions of dissolving the resin and the raw material, but also has great effluence on the flow pattern, the aridity, the operability of the coating material, and the gloss of the coating film. The solvent and the diluent include water, or toluene, xylene, and butyl acetate. The content of the solvent and the diluent is 10-90 wt %.

When the quaternary ammonium salt antibacterial is applied in a coating material, the coating material can be coated on any article having antibiotic requirement. The substrates to be coated include a plastic plate, a glass substrate, a cement substrate, and so on. Since the quaternary ammonium salt is likely to combine with the negative ions of the bacteria due to the positive ion nature thereof, thus having a high bactericidal power. Therefore, the objects coated by the coating material containing such quaternary ammonium salt antibacterial have an excellent antibiotic efficacy and a considerably persistent bacteriostatic effect.

In addition to the coating material, the antibacterial of the present invention can also be applied in lotions.

### EXAMPLES

### Test of the Stability of Antibacterial in Latex Paint

Four quaternary ammonium salt antibacterials A, B, C, and D shown in table 1 were prepared respectively, in which A was a quaternary ammonium salt antibacterial having the siloxane structure, and the other three antibacterials B, C, and D were quaternary ammonium salt antibacterials having no siloxane structure. Then, the four antibacterials A, B, C, and D were respectively added into a latex paint, and formulated to have a concentrations of 500, 1500, 2500, and 5000 ppm, and the pH value was adjusted to about 7. Changes of the antibacterials in the latex paint were observed. The results are listed in Table 2. The results in Table 2 show that the quaternary ammonium salt antibacterial A having the siloxane structure exhibit good miscibility in the latex paint.

TABLE 1

| Antibacterial | structure |
|---|---|
| A | 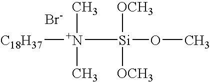 |
| B | 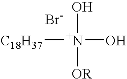 |
| C | 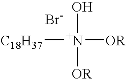 |
| D | 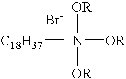 |

TABLE 2

| Content of | | | | |
|---|---|---|---|---|
| Antibacterial | | | | |
| (ppm) | A | B | C | D |
| 500 | miscible | precipitate | precipitate | precipitate |
| 1500 | miscible | precipitate | precipitate | precipitate |
| 2500 | miscible | precipitate | precipitate | precipitate |
| 5000 | miscible | precipitate | precipitate | precipitate |

### Potential Measurement

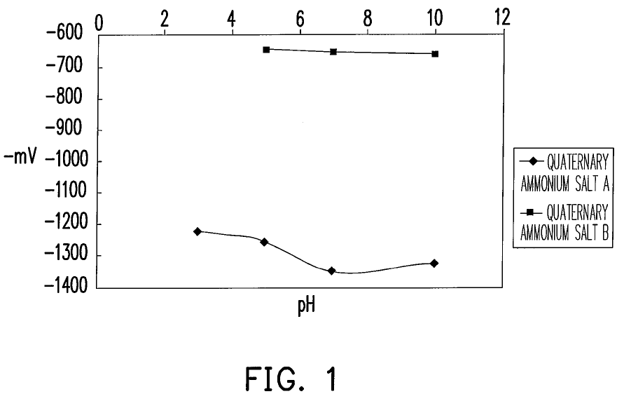

The methanol solution including 45% of the organic quaternary ammonium salt A having the siloxane structure and the methanol solution including 45% of the organic quaternary ammonium salt Bhaving no siloxane structure were respectively adjusted to a pH value of 0-7 by 0.1 N HCl and 0.1 N NaOH, and formulated to have a concentration of about 5%. The surface potentials were measured with Zetasizer, Nano ZS (Malvern, United Kingdom). The results are shown in FIG. 1. It is found from FIG. 1 that, the electronegativity of the organic quaternary ammonium salt A having the siloxane structure is increased with the increment of pH value; while the electronegativity of the organic quaternary ammonium salt B having no siloxane structure will not change with the pH value. The results show that adjusting the pH value can effectively change the charges of the organic quaternary ammonium salt having the siloxane structure.

### Antibiotic Experiment

After formulating the organic quaternary ammonium salt having the siloxane structure to have a concentration of 5% and adjusting the pH value to be 7, an antibiotic experiment was performed according to Japanese Standards Testing Method (JISZ 2801-2000). The results are listed in Table 3. It is indicated by the results in Table 3 that, the bacteriostatic ratio of the quaternary ammonium salt antibacterial of the present invention is greater than 99.99%.

TABLE 3

| | Bacteria count | Bacteria count | Bacteria count |
|---|---|---|---|
| Test Item | at 0 h * 3 sheets | at 3 h * 3 sheets | at 24 h * 3 sheets |
| Control Group (neat | 1.11 × 10⁶ | 1.31 × 10⁶ | 7.7 × 10⁵ |
| cement paint) | | | |
| Quaternary | 3.73 × 10⁵ | 1.48 × 10³ | <10 |
| Ammonium Salt | | | |
| having the siloxane | | | |
| structure | | | |
| Antimicrobial | 0.47 | 2.95 | 4.89 |
| Activity Value | | | |
| Bacteriostatic Ratio | less than 90% | 99% | 99.99% |

As described above, the quaternary ammonium slat antibacterial of the present invention can be dissolved in paints, lotions or surfactants having negative ions, to exert antibacterial effect efficiently.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A quaternary ammonium salt antibacterial, the structure of the quaternary ammonium salt antibacterial is shown as below: 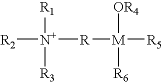
wherein M includes Si;
R represents a single bond or a C₁-C₄ alkyl group;
R₁-R₃ are the same or different to each other and represent a C₃-C₁₈ alkyl group, respectively;
R₄ represents a C₁-C₈ alkyl group or hydrogen;
R₅-R₆ are the same or different to each other and represent a C₁-C₈ alkyl group, alkoxy group, or hydroxy group, respectively; and
X represents a halogen.

2. A lotion which contains the quaternary ammonium salt antibacterial as claimed in claim 1.

3. A water-borne coating material having antibiotic property, comprising:
10-90 wt % of resin;
10-90 wt % of water; and
10-20000 ppm quaternary ammonium salt antibacterial, the structure of the quaternary ammonium salt antibacterial is shown as below: 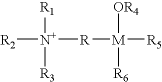
wherein M represents Si;
R represents a single bond or a C₁-C₄ alkyl group;
R₁-R₃ are the same or different to each other and represent a C₃-C₁₈ alkyl group, respectively;
R₄: represents a C₁-C₈ alkyl group or hydroxyl group;
R₅-R₆ are the same or different to each other and represent a C₁-C₈ alkyl group, alkoxy group, or hydrogen, respectively; and
X represents a halogen.

4. The water-borne coating material having antibiotic property as claimed in claim 3, further includes an additive, the content of the additive is about 0.1-50 wt %.

5. The water-borne coating material having antibiotic property as claimed in claim 3, wherein the pH value of the water-borne coating material is about 2-9.
